(19) United States
(12) Plant Patent Application Publication
(10) Pub. No.: US 2021/0259145 P1
(43) Pub. Date: Aug. 19, 2021

(54) Geranium Plant Named 'MACGER001'

(50) ;

(71) Applicants: Alasdair MacGregor, Kirkcudbright (GB); Elizabeth MacGregor, Kirkcudbright (GB)

(72) Inventors: Alasdair MacGregor, Kirkcudbright (GB); Elizabeth MacGregor, Kirkcudbright (GB)

(21) Appl. No.: 17/178,448

(22) Filed: Feb. 18, 2021

(30) Foreign Application Priority Data
Feb. 18, 2020 (QZ) 2020/0533

(51) Int. Cl. A01H 6/42

(52) U.S. Cl. PLT/325

(57) ABSTRACT

‘MACGER001’ is a new and distinctive variety of Geranium hybrid plant which is characterized by dark greyed-green foliage that is suffused with bronze and vibrant red-purple flowers with very dark purple venation and floral eye. The new variety propagates successfully by stem cuttings and tissue culture and has shown to be uniform and stable in the resulting generations from asexual propagation.

### CROSS REFERENCE TO RELATED APPLICATIONS

This application claims priority to the Community Plant Variety Rights application number 2020/0533, filed Feb. 18, 2020, which is herein incorporated by reference.

Latin name of the genus and species: The Latin name of the genus and species of the novel variety disclosed herein is Geranium hybrid.

Variety denomination: The inventive variety of Geranium hybrid disclosed herein has been given the variety denomination ‘MACGER001’.

### BACKGROUND OF THE INVENTION

Parentage: ‘MACGER001’ is the result of a controlled cross-pollination breeding program carried out by the inventors at their commercial nursery in Kirkcudbright, Scotland, in the summer of 2015. The inventors performed a controlled cross-pollination of the emasculated seed parent, an unnamed Geranium pratense plant (unpatented), with an unnamed Geranium psilostemon plant (unpatented), the pollen parent. Said pollination was performed in an insect proof environment. Seed from said cross was harvested, then germinated, and the resulting seedlings were then grown to a mature size in a protected poly tunnel greenhouse. In October of 2015, the inventors selected the new Geranium cultivar for its unique flower color and dark foliage. This new and distinctive cultivar was given the name ‘MACGER001’.

Asexual Reproduction: Asexual reproduction of ‘MACGER001’ was first accomplished in October of 2015 by way of vegetative division and subsequently by way of meristematic tissue culture micro propagation at a laboratory in Rijswijk, the Netherlands. Four successive generations have shown that the unique features of the instant cultivar are stable and reproduce true to type.

### SUMMARY OF THE INVENTION

The cultivar ‘MACGER001’ has not been observed under all possible environmental conditions and the phenotype may vary somewhat with variations in the instant environment such as temperature, day length, and light intensity, without, however, any variance in genotype. The following traits have been repeatedly observed and represent the distinguishing characteristics of the new Geranium cultivar, ‘MACGER001’.
1. Geranium ‘MACGER001’ exhibits dark greyed-green foliage that is suffused with a dark bronze coloration; and
2. Geranium ‘MACGER001’ exhibits foliage that is margined greyed-purple; and
3. Geranium ‘MACGER001’ exhibits vibrant red-purple flowers with very dark purple veins radiating outward from a very dark purple floral eye.

### BRIEF DESCRIPTION OF THE FIGURES

FIG. 1 shows, as nearly true as it is reasonably possible to make the same in color illustrations of this type, the typical foliage and growth characteristics of the new cultivar, ‘MACGER001’.

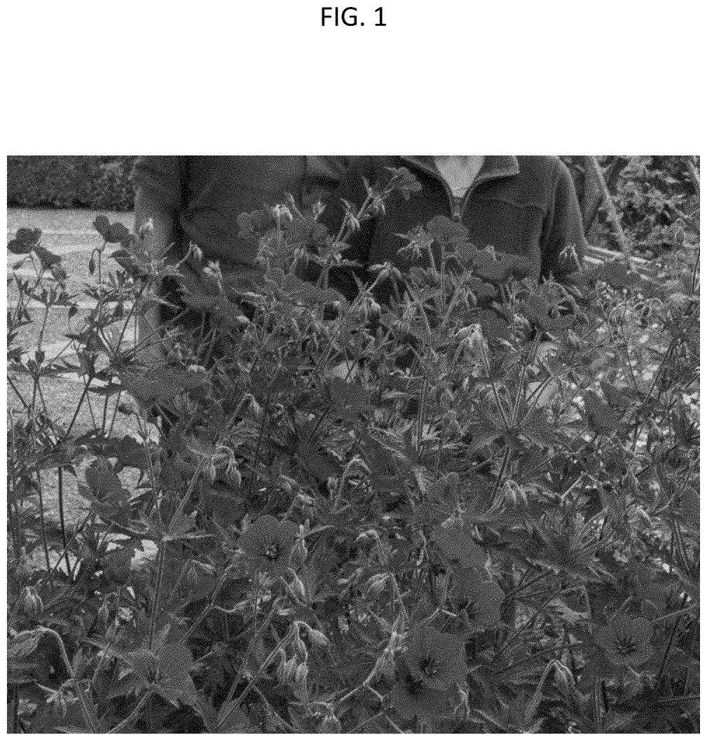

FIG. 2 shows, as nearly true as it is reasonably possible to make the same in color illustrations of this type, the typical flower of the plant in FIG. 1.

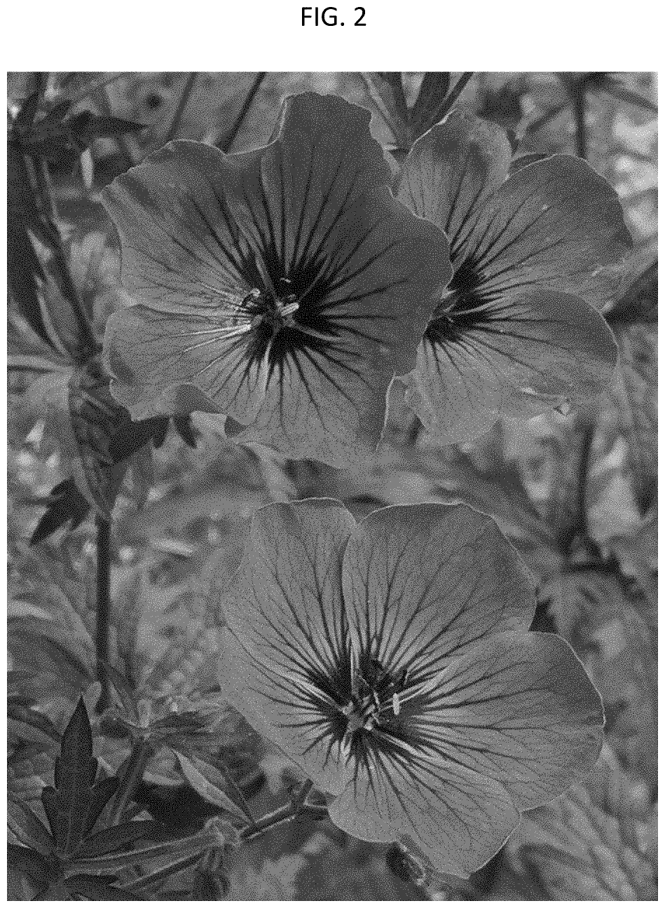

### BOTANICAL DESCRIPTION OF THE PLANT

A detailed botanical description of a new and distinct variety of Geranium known as ‘MACGER001’ is not yet available. A comparison with the parents and most similar variety of common knowledge are provided below.

A botanical description of ‘MACGER001’ and comparisons with the parents and most similar variety of common knowledge are provided below.
General plant description: Botanical description is not yet available.
Root system: Botanical description is not yet available.
Stem: Botanical description is not yet available.
Foliage: Botanical description is not yet available.
Inflorescence: Botanical description is not yet available.
Flower buds: Botanical description is not yet available.
Flower: Botanical description is not yet available.
Reproductive organs: Botanical description is not yet available.
Seed and fruit: Botanical description is not yet available.
Comparisons with the parent plants: Plants of the new cultivar ‘MACGER001’ differ from its seed parent, an unnamed Geranium pratense (not patented), by the characteristics described in Table 1.

TABLE 1

| Characteristic | ‘MACGER001’ | The seed parent. |
|---|---|---|
| General | Lighter green by | Darker green by |
| coloration of | comparison to | comparison to |
| the foliage. | the seed parent, | ‘MACGER001’. |
| | and suffused with | |
| | a bronze coloration. | |
| General | Darker red-purple | Lighter red-purple |
| coloration of | by comparison to | by comparison to |
| the flower. | the seed parent. | ‘MACGER001’. |
| General | Darker purple | Lighter purple by |
| coloration of | by comparison | comparison to |
| the flower eye. | to the seed parent. | ‘MACGER001’. |

Plants of the new cultivar ‘MACGER001’ may be distinguished from its pollen parent, an unnamed Geranium psilostemon plant (not patented), by the characteristics described in Table 2.

TABLE 2

| Characteristic | ‘MACGER001’ | The pollen parent |
|---|---|---|
| General | Darker green by | Lighter green by |
| coloration of | comparison to | comparison to |
| the foliage. | the pollen parent, | ‘MACGER001’. |
| | and suffused with | |
| | a bronze coloration. | |
| General | Lighter red-purple | Darker red-purple by |
| coloration of | by comparison | comparison to |
| the flower. | to the pollen parent. | ‘MACGER001’. |
| General | Darker purple by | Lighter purple by |
| coloration of | comparison to | comparison to |
| the flower eye. | the pollen parent. | ‘MACGER001’. |

Comparison with the most similar variety of common knowledge: Plants of the new cultivar ‘MACGER001’ may be distinguished from the most similar known commercial comparator, plants of the species Geranium psilostemon (unnamed and unpatented), by the characteristics described in Table 3.

TABLE 3

| | | Geranium |
|---|---|---|
| Characteristic | ‘MACGER001’ | psilostemon |
| Plant size. | Shorter than | Taller than |
| | Geranium | ‘MACGER001’. |
| | psilostemon. | |
| Foliage size. | Larger than | Smaller than |
| | Geranium | ‘MACGER001’. |
| | psilostemon. | |
| General | Greyed-green, | |
| coloration of | suffused with | Green. |
| the mature | a bronze | |
| foliage. | coloration. | |

That which is claimed is:

1. A new and distinct variety of Geranium hybrid plant named ‘MACGER001’, substantially as described and illustrated herein.
